(19) United States
(12) Patent Application Publication
(10) Pub. No.: US 2024/0164647 A1
(43) Pub. Date: May 23, 2024

(54) DEVICE AND METHOD FOR DETERMINING A PROTECTION FACTOR

(71) Applicant: COURAGE + KHAZAKA ELECTRONIC GMBH, Köln (DE)

(72) Inventors: Georg WIORA, Köln (DE); Carina REBLE, Berlin (DE); Diana KHAZAKA-OHMANN, Köln (DE); Georg KHAZAKA, Köln (DE)

(73) Assignee: COURAGE + KHAZAKA ELECTRONIC GMBH, Köln (DE)

(21) Appl. No.: 18/515,292

(22) Filed: Nov. 21, 2023

(30) Foreign Application Priority Data
Nov. 21, 2022 (DE) 102022130758.1
Mar. 27, 2023 (DE) 102023107705.8

(51) Int. Cl. A61B 5/00; A61K 49/00; G01N 21/33

(52) U.S. Cl. CPC A61B 5/0075; A61B 5/445; A61K 49/0006; G01N 21/33

(57) ABSTRACT

Described herein is a protection factor evaluation system for determining a protection factor of a skin protection agent with a radiation source with exactly one LED, a detector unit with exactly one photodiode, a control unit and an evaluation unit. Furthermore, the invention relates to a method for determining a sun protection factor of a skin protection agent with the method steps of emitting radiation from precisely one LED of a radiation source, detecting remitted radiation with precisely one photodiode of a detector unit and evaluating the protection factor in an evaluation wavelength range, wherein the protection factor of the protection agent is evaluated from the radiation and a transmission spectrum, and wherein the data of the transmission spectrum for determining the protection factor are in silico and/or in vitro data.

### CROSS-REFERENCE TO RELATED APPLICATIONS

Benefit is claimed to German Patent Application No. DE 10 2022 130 758.1, filed Nov. 21, 2022, and German Patent Application No. DE 10 2023 107 705.8, filed Mar. 27, 2023. The contents of the foregoing applications are incorporated by reference herein in their entirety.

### FIELD

The invention relates to a protection factor evaluation system for determining a protection factor of a skin protection agent with a radiation source with exactly one LED, a detector unit with exactly one photodiode, a control unit and an evaluation unit. Furthermore, the invention relates to a method for determining a sun protection factor of a skin protection agent with the method steps of emitting radiation from precisely one LED of a radiation source, detecting remitted radiation with precisely one photodiode of a detector unit and evaluating the protection factor in an evaluation wavelength range, wherein the protection factor of the protection agent is evaluated from the radiation and a transmission spectrum, and wherein the data of the transmission spectrum for determining the protection factor are in-silico and/or in vitro data.

### BACKGROUND

The methods approved to date by the authorities of the European Union (EU) and the American Food and Drug Administration (FDA) for the determination of the SPF (Sun Protect Factor) are all harmful to the test persons involved, as they cause erythema, i.e. an inflammatory reaction of the skin caused by light (COLI PA-15 European Cosmetic, Toiletry and Perfumery Association: Colipa SPF Test Method 94/289, 1994; ISO standards 24442, 24443, 24444). Therefore, both the FDA and the EU have repeatedly pointed out that future research activities must be directed towards new methods for characterising the protective effect of sunscreen products in order to avoid late effects for the test subjects (European Commission, 20 Standardisation Mandate Assigned To CEN Concerning Methods For Testing Efficacy Of Sunscreen Products, M/389 EN, Brussels, 12 Jul. 2006).

The present invention is intended to fulfil this task. The existing methods are defined in various references:
Procedures defined in standards and regulations:
a. ISO 24444 defines a method for the in vivo determination of the SPF. The method is based on the generation of erythema on the skin of test subjects by radiation in the UVB range. The method is therefore harmful to the test person.
b. ISO 24443 defines an in vitro method for determining the UVA protection factor (UVAPF). The sunscreen is applied to a plastic plate so that a transmission spectrum of the sunscreen can be measured. Due to uncontrollable fluctuations in the procedure, the transmission spectrum is adapted to the result of the erythema test according to ISO 24444 by scaling and is therefore dependent on its performance. The plastic plate used is an unrealistic skin model with a roughened surface.
c. ISO 24442 defines an in vivo method in which the UVA protection factor is determined by means of the minimum UVA dose to produce irreversible pigmentation (sun tan) of the skin. This method also requires a change in the skin of the test person.
Patented processes:

DE 198 28 497 A1 describes a method in which, as in ISO 24444, erythema is produced in test subjects by UV irradiation of the skin. In contrast to ISO 24444, these are detected by reflectance spectroscopy. The method is therefore also harmful. The optical effect (protection) of the sunscreen is not recorded by direct optical measurements, but via a biological reaction of the body.

DE 10 2004 020 644 A1 describes a method in which the generation of radicals by UV exposure is quantitatively measured in vivo using electron spin resonance (ESR). Here, too, the optical effect of the sunscreen is only recorded indirectly. In addition, the measurement of ESR is technically complex and requires relatively large, stationary devices (desktop devices). They are also sensitive to interference from high-frequency radiation or rapid temporary magnetic field changes, e.g. from electrical switching processes.

In order to determine the label SPF of topically applied sunscreens in vivo, test methods such as ISO 24444, the FDA guideline or the Australian standard are used worldwide. The basis of all these methods is the induction of an erythemal skin reaction by irradiating the skin with UV light. This is necessary to determine the minimum erythemal dose of untreated (MEDu) and product-treated skin (MEDp). Reliable in vitro methods, in which human skin is replaced by synthetic substrate carriers, are not available for SPF determination.

Monochromatic devices are known to use conventional xenon lamps and are therefore expensive to purchase and operate. The monochromators used measure different wavelengths one after the other, which is unfavorable when the test subjects move. Polychromatic devices also use xenon lamps. The in vivo measured value is weighted by means of a filter so that it corresponds to the in vivo UVA-PF. Multi-LED devices for test institutes are another variant, but are significantly larger and more expensive due to the spectroscopic detection.

### SUMMARY

It is therefore the task of the invention to provide a method for determining a protection factor of a skin protection agent, which reduces the exposure due to the irradiation of the human skin, provides high-quality analysis results and can be carried out quickly and easily at the same time. It is also the task of the invention to provide a protection factor evaluation system for analyzing the sun protection factor of sunscreen compositions, which provides high-quality analysis results, reduces the exposure due to irradiation of the human skin and is inexpensive to manufacture and operate.

The task is solved by means of the protection factor evaluation system for determining a protection factor of a skin protection agent according to claim 1. Further advantageous embodiments of the invention are set out in the depending claims.

The protection factor evaluation system according to the invention for determining a protection factor of a skin protection agent has a radiation source with exactly one LED. According to the invention, the measurement and detection of the electromagnetic radiation is not carried out with a sum spectrum generated by different radiation sources, but with exactly one radiation source, the radiation source being an LED. As a result, the protection factor evaluation system according to the invention is significantly more compact and less expensive than known systems.

In addition, the protection factor evaluation system has a detector unit with exactly one photodiode. The detector unit comprises, for example, a monochromator, filter, photomultiplier, spectrometer and/or a photodiode. All of these devices are suitable for detecting electromagnetic radiation and recording its intensity depending on the wavelength. A photodiode is used to detect an in vivo measurement. As a result, the protection factor evaluation system according to the invention is more compact and less expensive than known systems.

The protection factor evaluation system also has a control unit and an evaluation unit. The detection window and the resolution of the spectrometer are determined by means of the control unit, the detected signals are stored, processed (e.g. amplified) and displayed by means of the control unit. The evaluation unit is, for example, a computer and has a suitable computer program for determining a protection factor.

In an advantageous further development of the invention, the radiation source and the detector unit are combined in a first structural unit. Further components, e.g. an evaluation unit, are not arranged in the first unit. The dimensions and mass of the first structural unit are limited by this advantageous design, compact and so small that a user can carry, hold and operate the first structural unit with both hands, preferably with one hand, without further aids.

In a further embodiment of the invention, the first structural unit has a first communication unit. By means of the first communication unit, the data of the remitted and/or transmitted electromagnetic radiation recorded by the photodiode can be sent to the evaluation unit and data can be received from the external evaluation unit. The protection factor of a protective device can be determined by means of the evaluation unit.

In a further embodiment of the invention, the first structural unit and the control unit or the evaluation unit are arranged in different housings. In a further embodiment of the invention, the different housings have no structural connection to one another. The housings protect the respective components from the effects of the weather. At the same time, the first structural unit, control unit and evaluation unit can be arranged in housings that are each arranged independently of one another, whereby the individual components are resilient to damage and/or software failures.

In a further embodiment of the invention, the first structural unit has the control unit. By means of the first control unit, which is also arranged in the first structural unit, the first structural unit can be controlled, and the radiation detected by the photodiode can also be processed by means of the first control unit. The detection window and the resolution of the detection unit are determined by means of the first control unit, and the detected signals are stored and processed (e.g. amplified).

In a further embodiment of the invention, the evaluation unit is arranged in a second structural unit. The evaluation unit in the second structural unit is advantageously arranged at a spatial distance from the first structural unit. By means of the first communication unit, which is connected to the first control unit, the data of the remitted and/or transmitted electromagnetic radiation recorded by the photodiode can be sent to the evaluation unit and data can be received from the external evaluation unit. The protection factor of a protective device can be determined by means of the evaluation unit.

In a further embodiment of the invention, the second structural unit has a second communication unit. In a further embodiment of the invention, the first communication unit and the second communication unit are suitable for communicating with each other. In a further embodiment of the invention, the first and second communication units are adapted to communicate with each other wirelessly. The wireless connection between the first and second communication units enables flexible positioning of the first structural unit and the second structural unit independently of each other. In a further embodiment of the invention, the first and/or the second communication unit are suitable for communicating with each other via a public network. In a further embodiment of the invention, the public network is a mobile phone network, a WLAN, NFC network and/or a Bluetooth connection. The first and second communication units therefore utilize existing networks with established interfaces.

In a further embodiment of the invention, the LED is suitable for emitting light with a wavelength in the UVA range (irradiation wavelength 320 nm to 400 nm). The radiation is preferably emitted in vivo on the human skin in accordance with ISO 24442. ISO 24442 defines an in vivo method in which the UVA protection factor is determined by means of the minimum UVA dose to produce irreversible pigmentation (sun tan) of the skin.

In an advantageous further development of the invention, the first structural unit is a hand-held device. A hand-held device in the sense of the invention is a device that is easy and comfortable to hold and operate, even over a longer period of time. The hand-held device has such dimensions and such a mass that a user can carry, hold and operate the hand-held device with both hands. Preferably, the hand-held device has such dimensions and such a mass that a user can carry, hold and operate the hand-held device with one hand.

The first structural unit only has the components for generating and detecting radiation and controlling them. Further components, e.g. an evaluation unit, are not arranged in the hand-held device. The dimensions and mass of the first structural unit are limited by this advantageous design, compact and so small that a user can carry, hold and operate the first structural unit with both hands, preferably with one hand, without further aids. The mass of the first structural unit is at most 1000 g, preferably at most 500 g and particularly preferably at most 200 g. The first structural unit is therefore suitable for a user to transport the first structural unit comfortably and to carry out multiple determinations of a protection factor of a skin protection agent, even over a longer period of time.

In a further aspect of the invention, the second structural unit is a mobile device and preferably a smartphone. The mobile device can also be a notebook computer, tablet or the like and enables flexible positioning of the first structural unit and the evaluation unit independently of one another. The mobile device or smartphone has a suitable application for determining the protection factor of a protective means.

The task is also solved by means of the method according to the invention for determining a protection factor of a skin protection agent. Further advantageous embodiments of the invention are also set out in the dependent claims.

The method according to the invention for determining a protection factor of a skin protection agent has three method steps: In the first method step, radiation is emitted from precisely one LED of a radiation source. The LED generates electromagnetic radiation with an irradiation wavelength from the blue spectral range (from about 400 nm to 500 nm wavelength) and/or the UV range (280 nm to 400 nm).

The wavelength range is defined as follows:
280-320 nm (UVB) with <0.1% of the total UV intensity,
320 nm to 340 nm (UVA II) 8% to 20% of the total UVA intensity
340 nm to 400 nm (UVA I) 80% to 92% of the total UVA intensity
400 nm to 500 nm, blue light

In the second process step, remitted radiation is detected by exactly one photodiode of a detector unit. The ratio of the intensities of the remitted radiation to the radiation coupled into the measuring body is a measure of the protective capability of the protective means. Like the irradiation wavelength, the detection wavelength preferably comprises a wavelength range, whereby the wavelength range of the detection wavelength preferably lies within the range of the irradiation wavelength or comprises the entire range of the irradiation wavelength.

In the third process step, the protection factor is evaluated in an evaluation wavelength range, whereby the protection factor of the protective agent is evaluated from the radiation and a transmission spectrum. In addition, the data of the transmission spectrum for determining the protection factor are in silico and/or in vitro data. Due to the high absorption properties, human skin does not emit enough UVB radiation to measure the absorption spectrum of the applied product in the UVB range. It is therefore necessary to measure the absorption spectrum of the test material in the UVB part of the spectrum (280-320 nm) separately using a different technique. A transmission spectrum is determined based on the UV transmittance of protective films in vitro. The substrates to which the protective films are applied only approximate the inhomogeneous surface structure of human skin, such as polymethyl methacrylate (PMMA) sheets with a rough surface according to ISO 24443. The transmission spectrum data includes intensity versus wavelength in preferably digitized format. In a further embodiment of the invention, the transmission spectrum data is in-silico data. Such data of the transmission spectrum are therefore neither determined in vivo on a test subject nor in vitro according to ISO 24443, but are estimated or determined by calculation. If the properties of the filter substances of a protective agent are known, the transmission can be calculated and simulated. The simulated transmission can be used to calculate the sun protection factor and all variables that characterize the protection factor.

In a further development of the invention, data from the detected remitted radiation is transmitted to an evaluation unit. The evaluation unit is, for example, a computer and has a suitable computer program.

In a further embodiment of the invention, the evaluation unit is arranged in a different structural unit than the radiation source and/or the detector unit. In a further embodiment of the invention, the evaluation unit is a computer or a mobile radio device. The computer or mobile radio device is preferably arranged remotely from the radiation source and/or the detector unit and has a suitable computer program for determining a protection factor. The evaluation unit itself can therefore be a commercially available computer or a commercially available mobile phone, e.g. a smartphone.

In a further embodiment of the invention, the emitted radiation comprises an irradiation wavelength range between 280 nm and 2000 nm, preferably 280 nm to 800 nm and particularly preferably the range from 280 nm to 500 nm. In the context of this document, the LED is a technical device for generating electromagnetic radiation. An LED is therefore not an optical element for guiding, redirecting or changing the intensity and/or wavelength of electromagnetic radiation. A radiation source is therefore not, for example, an optical fibre, grating, prism or filter. The LED generates electromagnetic radiation with an irradiation wavelength between the blue spectral range (from about 400 nm to 500 nm wavelength) and the UV range (280 nm to 400 nm).

In a further embodiment of the invention, the evaluation wavelength range is different from the irradiation wavelength range. In a further embodiment of the invention, the evaluation wavelength comprises the wavelength range from 280 nm to 2000 nm, preferably the wavelength range from 280 nm to 800 nm or more preferably the wavelength range from 280 nm to 500 nm. In a further development, the evaluation wavelength comprises a wavelength range from 400 nm to 500 nm and preferably from 400 nm to 450 nm. Preferably, the wavelength range of blue light adjacent to the UVA range (up to 400 nm) is evaluated. To determine the protective capacity of the protective agent in the UVB wavelength range (<320 nm), this wavelength range can also be optionally evaluated.

In a further embodiment of the invention, the evaluation of the protective ability of the protective agent for light in a wavelength range from 400 nm to 500 nm is carried out in a separate process from the evaluation of the protective ability of the protective agent for light in a wavelength range from 280 nm to 400 nm. Due to the high absorption properties, the human skin does not emit enough UVB radiation to measure the absorption spectrum of the applied product in the UVB range. It is therefore necessary to measure the absorption spectrum of the test material in the UVB part of the spectrum (280-320 nm) separately using a different technique. For this purpose, the in vivo remission in the wavelength range from 320 nm to 400 nm is recorded with a photodiode, the wavelength range from 280 nm to the LED wavelength and the wavelength range from the LED wavelength to 500 nm with an in silico and/or in vitro transmission spectrum.

In a further development of the invention, the wavelength range of the transmission spectrum comprises the evaluation wavelength. The protective factor of the protective agent is evaluated from the in vivo remitted radiation and the in silico and/or in vitro transmission spectrum. Due to the high absorption properties, the human skin does not emit sufficient UVB radiation to measure the absorption spectrum of the applied product in the UVB range. It is therefore necessary to measure the absorption spectrum of the test material in the UVB part of the spectrum (280-320 nm) separately using a different technique. The approach used in this paper utilizes the in vivo evaluation of the absolute UVA absorption as measured by an in vivo measurement with the use of a calculated in silico and/or in vitro transmission spectrum to determine the protection factor of the protective agent for a user. The evaluation and hybridization of an in vivo reflectance value with an in silico and/or in vitro transmission spectrum is performed to obtain a complete UV spectrum so that the protection factors are calculated according to the formulae of the applicable standard (ISO 24443). For this purpose, the wavelength range of the transmission spectrum comprises the evaluation wavelength, which preferably covers the range from 280 nm to 500 nm.

In a further embodiment of the invention, the wavelength range of the irradiation wavelength or the wavelength range of the detection wavelength is smaller than the wavelength range of the evaluation wavelength. The wavelength ranges of the irradiation wavelength and the detection wavelength are preferably the same in a wavelength range of at most 320 nm to 400 nm. The wavelength range of the evaluation wavelength comprises a maximum range of 280 nm to 500 nm.

In a further development of the invention, the wavelength range of the irradiation wavelength and/or the wavelength range of the detection wavelength is less than 100 nm, preferably less than 50 nm and particularly preferably less than 25 nm. Therefore, only one radiation source is required to emit electromagnetic radiation which has a small wavelength range of the irradiation wavelength. In the same way, a detector that can detect a small wavelength range of the detection wavelength is required to record the remission spectrum. The radiation source and detector can therefore be inexpensive to manufacture and operate.

In a further embodiment of the invention, the irradiation wavelength and/or the detection wavelength comprises only light with wavelengths outside the wavelength range from 400 nm to 450 nm. To detect an in vivo measurement, the UVA wavelength range (320 nm -400 nm) in particular is coupled into the measuring body.

In a further embodiment of the invention, the protection factor of the protective agent is evaluated from the remitted radiation and a transmission spectrum. The protective factor of the protective agent is evaluated from the in vivo remitted radiation and the in silico and/or in vitro transmission spectrum. Due to the high absorption properties, the human skin does not emit sufficient UVB radiation to measure the absorption spectrum of the applied product in the UVB range. It is therefore necessary to measure the absorption spectrum of the test material in the UVB part of the spectrum (280-320 nm) separately using a different technique. The approach used in this paper utilizes the in vivo evaluation of the absolute UVA absorption as measured by an in vivo measurement with the use of a calculated in silico and/or in vitro transmission spectrum to determine the protection factor of the protective agent for a user. The evaluation and hybridization of an in vivo reflectance spectrum with an in silico and/or in vitro transmission spectrum is performed to obtain a complete UV spectrum so that the protection factors are calculated according to the formulae of the applicable standard (ISO 24443). For this purpose, the wavelength range of the transmission spectrum comprises the evaluation wavelength, which preferably covers the range from 280 nm to 500 nm.

In an advantageous further development of the invention, the radiation source and/or the detector unit are arranged in a hand-held device. A hand-held device in the sense of the invention is a device that is easy and comfortable to hold and operate, even over a longer period of time. The hand-held device according to the invention has such dimensions and such a mass that a user can carry, hold and operate the hand-held device with both hands. Preferably, the hand-held device according to the invention has such dimensions and such a mass that a user can carry, hold and operate the hand-held device with one hand.

The hand-held device is suitable for introducing electromagnetic radiation into a measuring body, preferably the human skin, by means of the LED. The radiation source is an LED. In addition, the hand-held device is suitable for detecting the remitted and/or transmitted electromagnetic radiation by means of the photodiode. The hand-held device can be controlled by means of the first control unit, and the radiation detected by the photodiode can also be processed by means of the first control unit. The detection window and the resolution of the detection unit are determined by means of the first control unit, and the detected signals are stored and processed (e.g. amplified).

The hand-held device according to the invention only has the components for generating and detecting radiation and controlling them. Further components, e.g. an evaluation unit, are not arranged in the hand-held device. The dimensions and mass of the hand-held device according to the invention are limited by this advantageous design, compact and so small that a user can carry, hold and operate the hand-held device with both hands, preferably with one hand, without further aids. The mass of the hand-held device is a maximum of 1000 g, preferably a maximum of 500 g and particularly preferably a maximum of 200 g. The hand-held device is therefore suitable for a user to transport the hand-held device comfortably and to carry out multiple determinations of a protection factor of a skin protection product, even over a longer period of time.

### BRIEF DESCRIPTION OF THE DRAWINGS

Examples of embodiments of the method according to the invention for determining a protection factor and of the protection factor evaluation system according to the invention are shown schematically in simplified form in the drawings and are explained in more detail in the following description.

It shows:

FIG. 1: Protection factor evaluation system with sample head, first structural unit and second structural unit

FIG. 2: Sample head with beam inlet and beam outlet

FIG. 3: Protection factor evaluation handset with a first control unit and an operating unit

FIG. 4: Protection factor evaluation handset with a first control unit, an operating unit and an output device

FIG. 5: Protection factor evaluation handset with a first control unit

FIG. 6: Protection factor evaluation handset with a first control unit and an operating unit

FIG. 7: Protection factor evaluation system, evaluation unit and first control unit arranged as a single unit within the mobile radio device

FIG. 8: Protection factor evaluation handset with a first control unit, an operating unit and an output device

FIG. 9: Protection factor evaluation handset with a first control unit, an operating unit, an output device and a second control unit

FIG. 10: Protection factor evaluation system, evaluation unit and first control unit arranged in a modular unit within the mobile radio device

FIG. 11: Procedure for carrying out an in vivo measurement to evaluate a sun protection factor with reference and calibration measurement

FIG. 12: Method for performing an in vivo measurement including an in silico and/or in vitro spectrum for evaluating a sun protection factor

### DETAILED DESCRIPTION

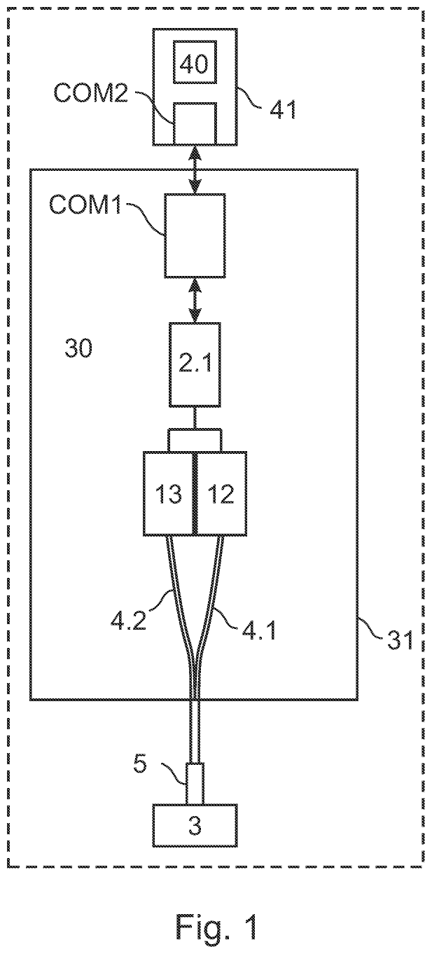

FIG. 1 schematically shows an embodiment example of the protection factor evaluation system 1 according to the invention for carrying out an in vivo measurement on the skin of a test subject 3. The protection factor evaluation system 1 has the first structural unit 30, which is arranged in the first housing 31. The first structural unit 30 has the radiation source device 12 and the detector unit 13. The radiation source device 12 comprises a single LED 12.1 as well as optical elements intended and/or suitable for conditioning and/or redirecting the first radiation generated by the first radiation source 12.1, e.g. light guide, filter, monochromator, mirror and/or other optical elements.

The first component 30 also has the detector unit 13, which has a single photodiode 13.1. Detector unit 13 and radiation source device 12 are connected via data lines to the first control unit 2.1, which in turn is connected to the evaluation unit 10 via the first communication unit COM1. The evaluation unit 10 is arranged externally from the first structural unit 30 in a second structural unit 40, the second structural unit 40 being arranged in a second housing 41. The first structural unit is connected to the sample head 5 via the optical fibres 4.1, 4.2.

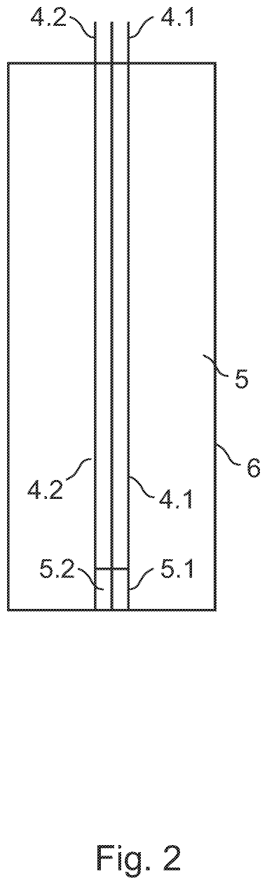

A sectional view of an embodiment of a sample head 5 is shown in FIG. 2. The sample head 5 has the beam outlet 5.1, with which the electromagnetic radiation generated by the measuring device 6 is irradiated into the human skin 3 via the light guide 4.1. The sample head 5 also has a beam inlet 5.2, with which the radiation remitted from the measuring location 3 is picked up and channeled into the detector unit 13 via the light guide 4.2.

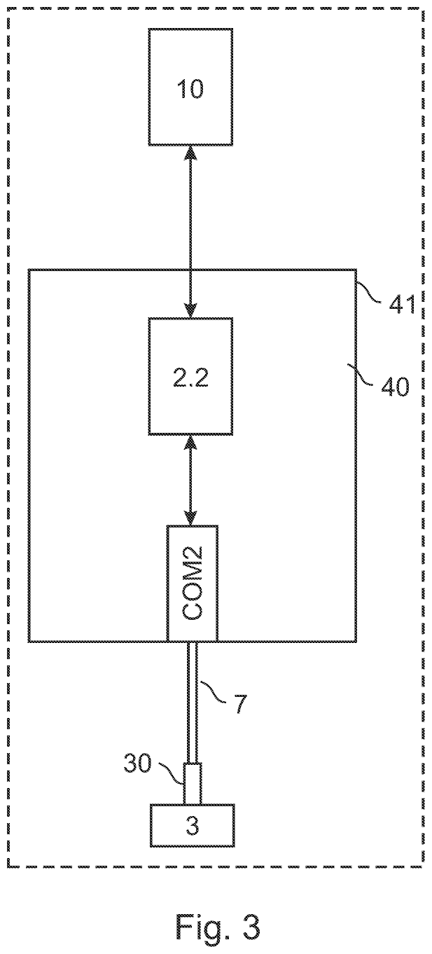

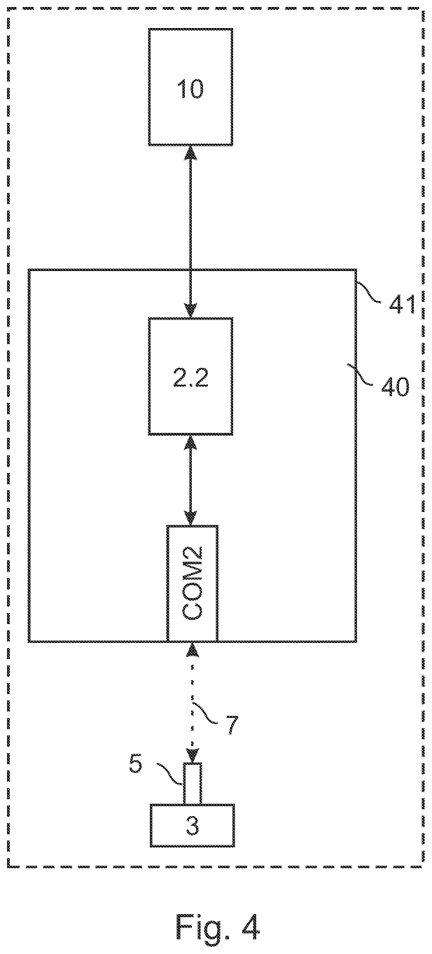

FIG. 3 and FIG. 4 show embodiments of the protection factor evaluation system 1 according to the invention. The protection factor evaluation system 1 has the hand-held device 30 (see FIG. 1), which is arranged directly on the measuring body 3 to determine a protection factor of a skin protection agent. The measuring body 3 is, for example, the skin of a test person. The hand-held device 30 has a first communication unit COM1 which is connected to a second communication unit COM2 of a mobile radio device 40 by means of a connection 7. The connection 7 is made by cable or a wireless Bluetooth connection; another connection option is an Internet connection.

The mobile communication device 40, in this embodiment example a smartphone with a suitable app, has a second control unit 2.2 which controls the method 400 for determining a protection factor. The second control unit 2.2 is connected to the second communication unit COM2, via which the data from the handset 30 is received. In this embodiment example, the evaluation unit 10 is arranged externally at a distance from both the mobile radio 40 and the handset 30. In this embodiment example, the evaluation unit 10 is a fixed desktop computer, but the evaluation unit 10 can also be a mobile device, e.g. also a smartphone, tablet or the like, and has a suitable app for determining a protection factor of a protective device.

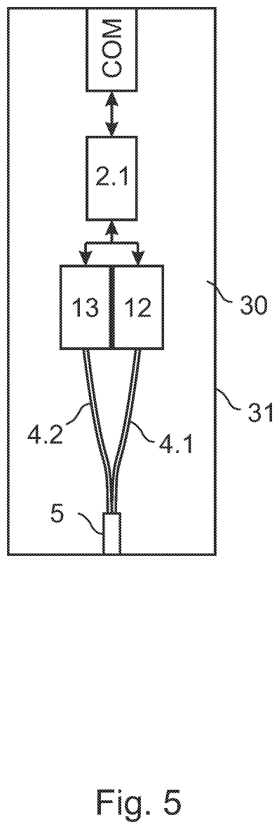

FIG. 5 shows an embodiment example of the hand-held device 30 according to the invention for performing an in vivo measurement. The hand-held device 30 has the radiation source device 12. The radiation source device 12 has an LED 12.1 as well as optical elements which are intended and/or suitable for conditioning and/or redirecting the radiation generated by the LED 12.1, e.g. light guides, filters, monochromators, mirrors and/or other optical elements.

The light emitted by the radiation source device 12 with an irradiation wavelength between 280 nm and 2000 nm is introduced into the measuring body 3 via the sample head 5 by means of a light guide 4.1. Preferably, the irradiation wavelength is in the range from 280 nm to 800 nm, particularly preferably in the range from 280 nm to 500 nm. In this and all other embodiments, the irradiation wavelength is in the range from 280 nm to 500 nm (UVA to blue light). The light emitted by the measuring body 3 reaches the detector unit 13 via a further light guide 4.2. The detector unit 13 can have a monochromator, filter, photomultiplier, spectrometer and/or a photodiode. In this and all subsequent embodiments, the detector unit 13 has a photodiode as detector 13.1.

Detector unit 13 and radiation source device 12 are connected to a first control unit 2.1, which in turn is connected to the first communication unit COM1 via a further data line. The first control unit 2.1 is usually a computer chip with a suitable computer program stored in a memory. The first communication unit COM1 is connected wirelessly to an externally arranged evaluation unit 10 via a second communication unit COM2 (not shown), whereby the first communication unit COM1 and the second communication unit COM2 communicate with each other. All the above-mentioned components of the hand-held device 30 are arranged in a first housing 31 that protects the components from soiling.

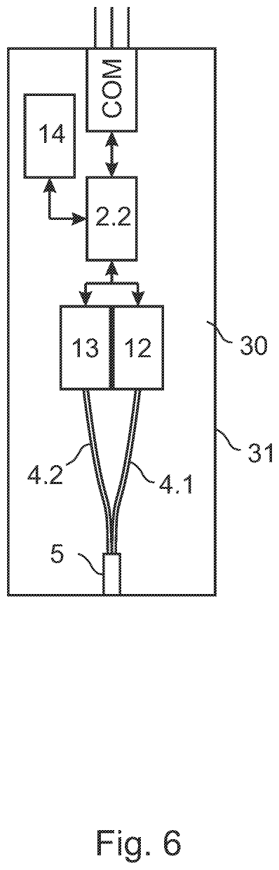

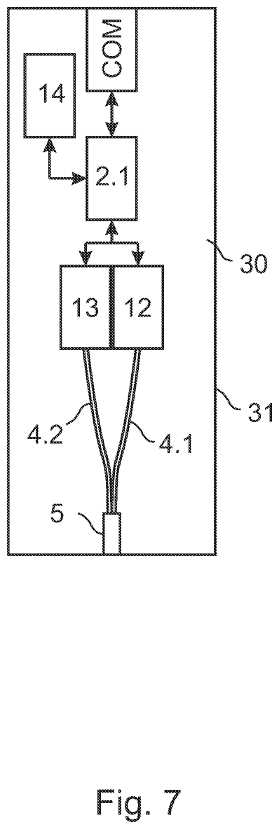

Variants of the hand-held device 30 are shown in FIG. 6 and FIG. 7. The hand-held device 30 shown here corresponds to the previous embodiment example (see FIG. 5), except that the first control unit 2.1 (2.2 in FIG. 7) is connected to an operating unit 14. By means of the control unit 14, a user of the hand-held device 30 can control the first control unit 2.1 and thus operate the hand-held device 30, e.g. generate or switch off the radiation, select the radiation wavelength range of the radiation source 12.1 and control the intensity of the radiation generated by the radiation source 12.1. First communication unit COM1 and second communication unit COM2 are connected to each other by means of a cable connection (FIG. 6) or wirelessly (FIG. 7).

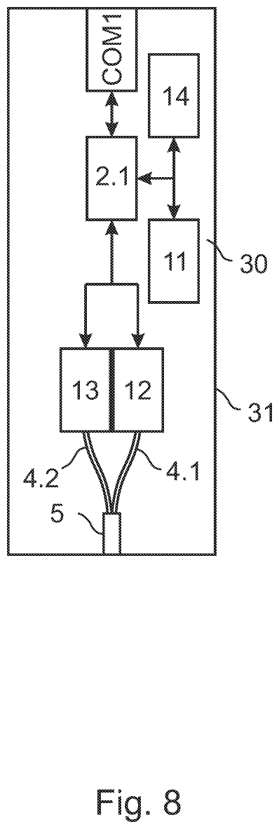

FIG. 8 shows a further embodiment example of the hand-held device 30. The hand-held device 30 shown here corresponds to the previous embodiment example (see FIG. 7); in addition, the first control unit 2.1 is connected to the operating unit 14 and an output device 11. In this embodiment example, the output device 11 is arranged in the hand-held device 30. The output device 11 can be used to indicate whether the LED 12.1 is emitting radiation. Alternatively or additionally, the parameters of the radiation to be emitted by the LED 12.1 and the photodiode 13.1 can be displayed. Furthermore, the specific protection factor of a skin protection agent can be displayed.

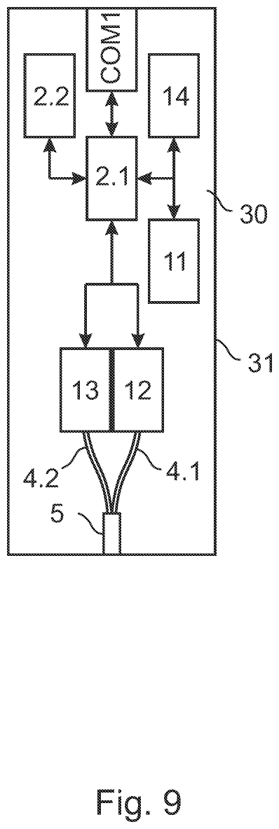

A preferred embodiment example of the hand-held device 30 is shown in FIG. 9. The hand-held device 30 shown here corresponds to the previous embodiment example (see FIG. 4); in addition, the hand-held device 30 has a second control unit 2.2, which is connected to the first control unit 2.1.

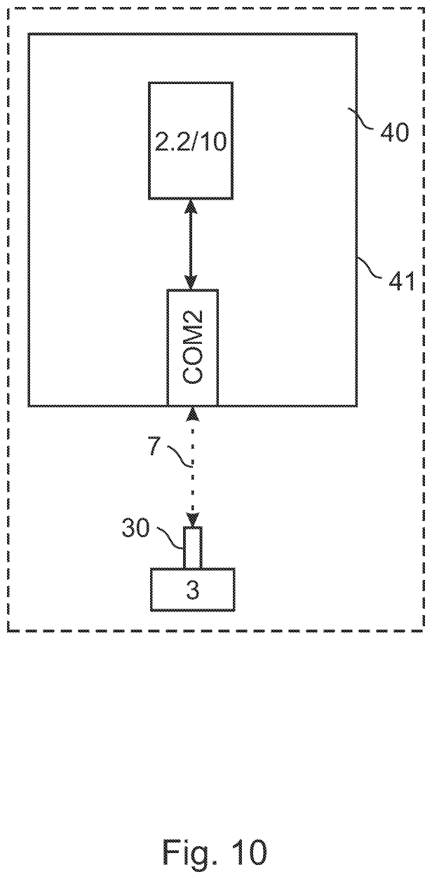

FIG. 10 shows a further embodiment example of the protection factor evaluation system 1 according to the invention. Like a previously shown embodiment example (see FIG. 3), the protection factor evaluation system 1 has the handset 30. The handset 30 has a first communication unit COM1, which is connected to a second communication unit COM2 of the mobile radio device 6. The mobile device 40 has a first control unit 2.2, which controls the method 400 for determining a protection factor. The first control unit 2.2 is connected to the communication unit COM1. In this embodiment example, the evaluation unit 10 is arranged in the smartphone 40 in a modular unit with the first control unit 2.2.

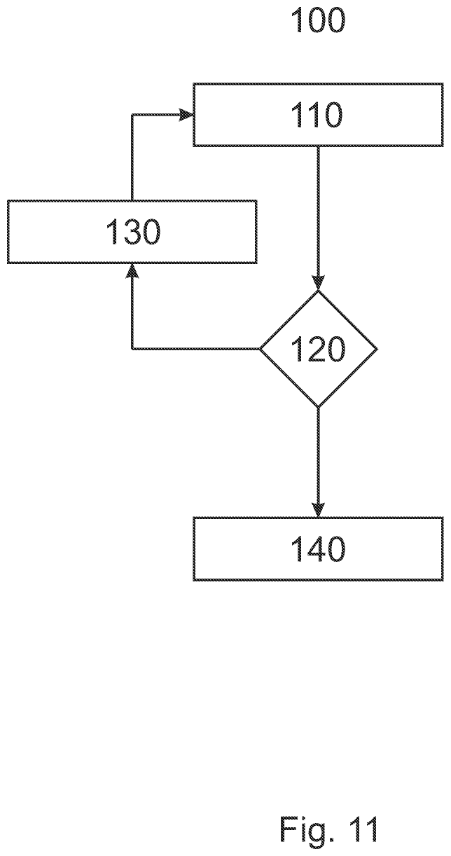

FIG. 11 shows an embodiment example of a procedure 100 of an in vivo measurement for recording the remission spectrum using the protection factor evaluation system 1 according to the invention, as shown in the previous embodiment examples (see FIG. 1 to FIG. 10). The measurement is carried out in vivo according to ISO 24442 or 24444. The procedure 100 of a measurement requires the recording of one remission each of the skin of test person 3 untreated with protective agent and the skin of test person 3 treated with protective agent.

The sample head 5 is applied to the untreated skin of test subject 3, i.e. the protective agent to be analyzed is not applied to the skin of test subject 3. For this purpose, a location on the inside of the forearm or the back of test subject 3 is usually selected. The first measurement 110 is then carried out by the first control unit 2.1 controlling the LED 12.1 in such a way that the light generated by the LED 12.1 is directed through the light guide 4.1 onto the skin of the test person 3. The electromagnetic radiation generated by the LED 12.1 has an irradiation wavelength range with an FWHM of up to 20 nm, whereby the electromagnetic radiation generated is in the wavelength range from 330 nm to 350 nm. The wavelength range of the detection wavelength, in which the photodiode 13.1 detects the remitted radiation by means of the light guide 4.2, also has a range of 20 nm in the range from 330 nm to 350 nm.

The light generated by the LED 12.1 is irradiated unfiltered onto the measuring body 3 in order to ensure a high S/R ratio. In particular, the light generated by the LED 12.1 is polychromatic with a maximum intensity at a wavelength in the UVA range of 340 nm. Alternatively, an LED 12.1 can be used that generates light with a maximum intensity in the UVA range of 365 nm. The irradiation occurs at an intensity that does not cause acute damage to the skin, which is below the simple MED or below the MZB values or significantly below the values caused by solar radiation. The light remitted by the skin of the subject 3 is transmitted through the light guide 4.2 to the photodiode 13.1 of the detector unit 13, detected by the photodiode 13.1 and converted into measured values, the measured values are sent to the first control unit 2.1 and stored in the first control unit 2.1.

The first control unit 2.1 then asks from 120 whether the second measurement of the skin of test person 3 treated with protective agent has already been carried out. If this is not the case, the first control unit 2.1 indicates this. To carry out the second measurement 110 with the protective agent applied, the protective agent is applied to the skin of test person 3 130, e.g. according to ISO 24442 or 24444 in the amount of 2.0 mg/cm² on the skin surface to be tested. The application 130 of the protective agent and the subsequent second measurement 110 are carried out on a comparable area of the test sample 3, in particular on the same area of the skin of a test subject 3, in order to ensure the reproducibility of the first and second measurements 110. Also, to ensure reproducibility, the first control unit 2.1 controls the LED 12.1 in such a way that the light generated by the LED 12.1 is guided through the light guide 4.1 onto the skin of the test subject 3, with the intensity and/or exposure time of the first and second measurements 110 being matched to one another. If the query 120 shows that the second measurement 110 has already taken place, the protective agent is evaluated 140. For this purpose, the evaluation unit 10 executes a program to calculate the reflectance spectrum T_{in vivo} according to equation 1:

$${T}_{in vivo} = \frac{1}{{SPF}_{in vivo}} = \sqrt{\frac{R}{{R}_{0}}} \qquad Eq . 1$$

with T_{in vivo} as a function of the wavelength λ, SPF_{in vivo} the protection factor determined by the in vivo method 100, R₁₀₀ reflected intensity of the untreated skin of test person 3 as a function of the wavelength λ, R reflected intensity of the skin of test person 3 treated with protective agent as a function of the wavelength λ. The method 100 described here requires a time of a few seconds to a few tens of seconds.

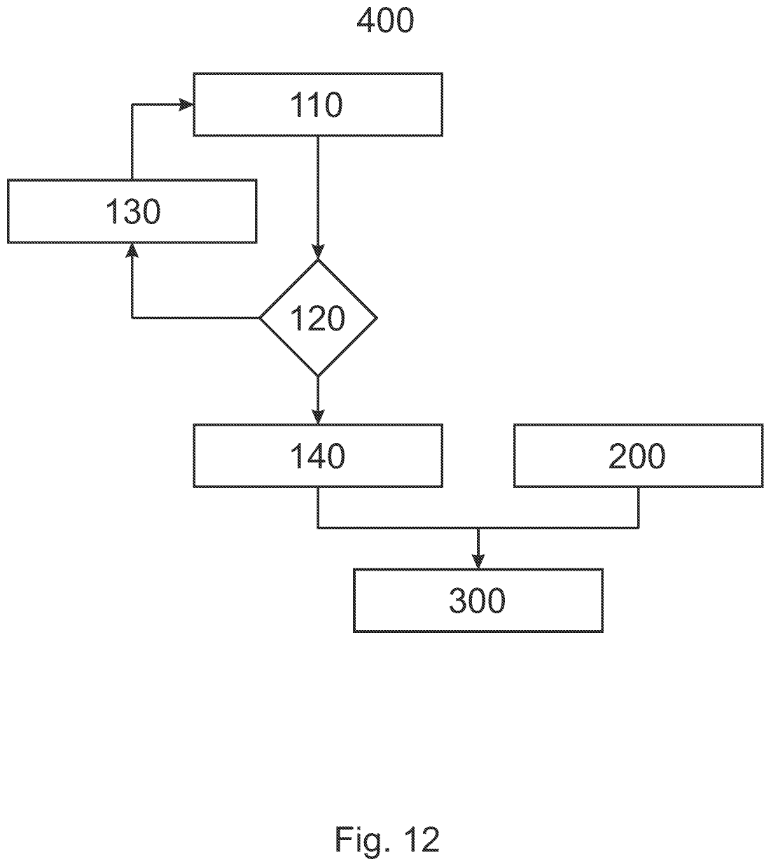

FIG. 12 shows an embodiment example of the method 400 according to the invention for determining a protection factor of a skin protection agent. Here, an in vivo reflectance measurement 100 (see FIG. 11) is combined with the data of a transmission spectrum 200. According to the invention, the values of the transmission spectrum 200 are calculated in silico and/or measured in vitro.

If the quantities and properties of the UV filter substances of a protective agent are known, the UV transmission can be calculated, considering the irregularity of the film and its photodegradation. The simulated UV transmission can be used to calculate in silico the protective capacity of the protective agent and all variables that characterize the protection against UVA and/or UVB radiation.

The in-silico determination of the data of a transmission spectrum is carried out, for example, in a laboratory based on ISO 24443. An application of 2.0 g/cm² of protective agent is assumed. In this example, the radiation emitted is in the wavelength range from 280 nm to 500 nm (UVB to blue light). In further embodiments, the irradiation wavelength range can be in the wavelength range between 280 nm and 2000 nm and preferably between 280 nm and 800 nm.

The evaluated wavelength range of the transmission spectrum comprises the wavelength range from 280 nm to 2000 nm, preferably the wavelength range from 280 nm to 800 nm or particularly preferably the wavelength range from 280 nm to 500 nm and/or the wavelength range from 400 nm to 500 nm and/or the wavelength range from 400 nm to 450 nm. In this embodiment example, the evaluation wavelength range of the transmission spectrum comprises 280 nm to 500 nm.

The data of the in silico and/or in vitro transmission spectrum is stored in a database and can be retrieved from the database at any time. To determine the protection factor of a skin protection product, the in silico and/or in vitro transmission spectrum is loaded into the evaluation unit 10 200, which is connected to the database via an internet connection.

The results of the evaluations of the in vivo emission spectrum 140 and the in silico and/or in vitro transmission spectrum are evaluated in combination by means of the evaluation unit 10 300. For this purpose, the hybrid transmission spectrum T_{hyb} is first calculated by means of a suitable computer program on the evaluation unit 10, whereby the in silico transmission spectrum T_{in silico} and/or in vitro transmission spectrum T_{in vitro} is scaled by means of the reflection spectrum T_{in vivo}:

$${T}_{h y b} ( λ ) = {T}_{in silico / in vitro} ( λ ) * \frac{{T}_{in vivo} ( {λ}_{L E D} )}{{T}_{in silico / in vitro} ( {λ}_{L E D} )} \qquad Eq . 3$$

The protective capability of the protective agent for the spectral range from UVA (320 nm) to the HEV spectral range (450 nm) SF is then calculated according to equation 4 (here E=IPD(λ) is the IPD spectrum; S=I(λ) is the solar spectrum):

$$S F = \frac{{\int }_{320}^{450} S ( λ ) E ( λ ) d λ}{{\int }_{320}^{450} S ( λ ) E ( λ ) {T}_{h y b} d λ} \qquad Eq . 4$$

The protective capability of the protective agent in the spectral range of blue light (400 nm to 500 nm) is determined according to equation 5 (here E=IPD(λ) is the IPD spectrum; S=I(λ) is the solar spectrum)

$$S F ( 400 nm - 500 nm ) = \frac{{\int }_{400}^{500} S ( λ ) E ( λ ) d λ}{{\int }_{400}^{500} S ( λ ) E ( λ ) {T}_{h y b} d λ} \qquad Eq . 5$$

The protective capability of the protective agent from 400 nm to 450 nm SF (400 nm-450 nm) is determined according to equation 6 (here E=IPD(λ) is the IPD spectrum; S=I(λ) is the solar spectrum):

$$S F ( 400 nm - 450 nm ) = \frac{{\int }_{400}^{450} S ( λ ) E ( λ ) d λ}{{\int }_{400}^{450} S ( λ ) E ( λ ) {T}_{h y b} d λ}$$

The protective capacity of the protective agent for the spectral range of UVA (320-400 nm) UVA-SF is then calculated according to equation 4 (here E=PPD(λ) is the PPD spectrum; S=I(λ) is the solar spectrum or UVA source for PPD-test):

$$UVA - SF = \frac{{\int }_{320}^{400} S ( λ ) E ( λ ) d λ}{{\int }_{320}^{400} S ( λ ) E ( λ ) {T}_{h y b} d λ} \qquad Eq . 7$$

The protective capacity of the protective agent for the spectral range from UVB to UVA (280-400 nm) UV-SF is then calculated according to equation 4 (here E (λ) is the erythema action spectrum; S (λ) is the spectrum of the sun simulator according to ISO 24444)

$$UV - SF = \frac{{\int }_{290}^{400} S ( λ ) E ( λ ) d λ}{{\int }_{290}^{400} S ( λ ) E ( λ ) {T}_{h y b} d λ} \qquad Eq . 8$$

In all equations for SF or UVA-SR, a correction function F (SF)=SF_korr or F (UVA-SF)=UVA-PF_korr is optionally used, which describes skin type-dependent differences, for example. A correction function makes values of different skin types comparable and outputs a corrected value SFₖₒᵣᵣ or UVA-PFₖₒᵣᵣ

For example, F can be a linear factor (i.e. F(SF)=SF*C) or an exponential function (i.e. F(SF)=SF^{C}). C can be dependent on the skin type or the ITA^{∘} value, for example. As shown in Eq. 4, the formulae in equations 2 to 8 can be written as follows:

$$S {F}_{korr} = F ( S F ) = F ( \frac{{\int }_{320}^{450} S ( λ ) E ( λ ) d λ}{{\int }_{320}^{450} S ( λ ) E ( λ ) {T}_{h y b} d λ} ) \qquad Eq . 9$$

As the in vivo value is measured without photodegradation, the photodegradation should be considered appropriately. This can be done, for example, by calculating T_{in silco} with (T_{in stico_irr}(λ) and without photodegradation and a spectral quotient is calculated from this (the same applies to T_{in vitro})

$$SRPD ( λ ) = \frac{{T}_{in silico\_irr} ( λ )}{{T}_{in silico} ( λλ )} . \qquad Eq . 10$$

This will then

T_{hyb_irr}(λ)=T_{hyb}(λ)*SRPD(λ) Eq.11

calculated.

The method can be calibrated using suitable reference procedures such as electron spin resonance spectroscopy.

### REFERENCE LIST

1 Protection factor evaluation system
2.1 First control unit
2.2 Second control unit
3 Measuring body
4.1, 4.2 Fibre optics/fibre bundles
5 Sample head
5.1 Outlet
5.2 Admission
6 Housing of the sample head
7 Connection protection factor evaluation handse—second unit
10 Evaluation unit
11 Dispensing device
12 Radiation source device
12.1 Radiation source/LED
13 Detector unit
13.1 Detector/photodiode
14 Control unit
30 First structural unit/protection factor evaluation handset
31 First housing
40 Second structural unit/mobile device
41 Second housing
100 Carrying out a reflection measurement
110 Carrying out a calibration measurement
120 Query
130 Application of a protective agent
140 Evaluation
200 Read in transmission spectrum
300 Determination of the protection factor
400 Method for determining a protection factor of a skin protection product
COM1 First communication unit
COM2 Second communication unit

We claim:

1. Protection factor evaluation system (1) for determining a protection factor of a skin protection product comprising
a radiation source (12) with
exactly one LED (12.1),
a detector unit (13) with
exactly one photodiode (13.1) and
a control unit (2.1),
an evaluation unit (10).

2. A protection factor evaluation system (1) for determining a protection factor of a skin protection agent according to claim 1,
characterized in that
the radiation source (12) and the detector unit (13) are combined in a first structural unit (30).

3. A protection factor evaluation system (1) for determining a protection factor of a skin protection product according to claim 1,
characterized in that
the first structural unit (30) has a first communication unit (COM1).

4. A protection factor evaluation system (1) for determining a protection factor of a skin protection agent according to claim 1,
characterized in that
the first structural unit (30) and the control unit (2.1) or the evalution unit (10) are arranged in different housings (31, 41).

5. A protection factor evaluation system (1) for determining a protection factor of a skin protection agent according to claim 4,
characterized in that
the different housings (31, 41) have no structural connection to each other.

6. A protection factor evaluation system (1) for determining a protection factor of a skin protection agent according to claim 1,
characterized in that
the first structural unit (30) has the control unit (2.1).

7. A protection factor evaluation system (1) for determining a protection factor of a skin protection agent according to claim 1,
characterized in that
the evaluation unit (10) is arranged in a second structural unit (40).

8. A protection factor evaluation system (1) for determining a protection factor of a skin protection agent according to claim 7,
characterized in that
the second structural unit (40) has a second communication unit (COM2).

9. A protection factor evaluation system (1) for determining a protection factor of a skin protection agent according to claim 8,
characterized in that
the first communication unit (COM1) and the second communication unit (COM2) are suitable for communicating with each other.

10. A protection factor evaluation system (1) for determining a protection factor of a skin protection agent according to claim 1,
characterized in that
the LED (12.1) is suitable for emitting light with a wavelength in the UVA range.

11. A protection factor evaluation system (1) for determining a protection factor of a skin protection agent according to claim 1,
characterized in that
the first structural unit (30) is a hand-held device.

12. A protection factor evaluation system (1) for determining a protection factor of a skin protection agent according to claim 1,
characterized in that
the second structural unit (40) is a mobile device and preferably a smartphone.

13. Method (400) for determining a protection factor of a skin protection agent, comprising the method steps:
Emitting radiation from exactly one LED (12.1) of a radiation source (12),
Detection of remitted radiation from exactly one photodiode (13.1) of a detector unit (13),
Evaluation (300) of the protection factor in an evaluation wavelength range,
whereby the protection factor of the protective agent is evaluated from the remitted radiation and a transmission spectrum (300),
wherein the data of the transmission spectrum for determining the protection factor are in silico and/or in vitro data.

14. A method (400) for determining a protection factor of a skin protection agent according to claim 13,
characterized in that
data of the detected remitted radiation is transmitted to an evaluation unit (10).

15. A method (400) for determining a protection factor of a skin protection agent according to claim 14,
characterized in that
the evaluation unit (10) is arranged in a different structural unit (30, 40) than the radiation source (12.1) and/or the detector unit (13).

16. A method (400) for determining a protection factor of a skin protection agent according to claim 13,
characterized in that
the emitted radiation covers an irradiation wavelength range between 280 nm and 2000 nm, preferably 280 nm to 800 nm and particularly preferably the range from 280 nm to 500 nm.

17. A method (400) for determining a protection factor of a skin protection agent according to claim 16,
characterized in that
the evaluation wavelength range is different from the irradiation wavelength range.

18. A method (400) for determining a protection factor of a skin protection agent according to claim 13,
characterized in that
the protection factor of the protective agent is evaluated from the remitted radiation and a transmission spectrum (300).

19. A method (400) for determining a protection factor of a skin protection agent according to claim 13,
characterized in that
the evaluation unit (10) is a computer or a mobile phone.

20. A method (400) for determining a protection factor of a skin protection agent according to claim 13,
characterized in that
the radiation source (12.1) and/or the detector unit (13) are arranged in a hand-held device (30).
